(19) United States
(12) Patent Application Publication
(10) Pub. No.: US 2023/0157964 A1
(43) Pub. Date: May 25, 2023

(54) A STABLE FOOD-GRADE MICROCAPSULE FOR THE DELIVERY OF UNSTABLE AND FOOD-INCOMPATIBLE ACTIVE INGREDIENTS TO FOOD PRODUCTS

(71) Applicant: Nuversys LTD., Upper Galilee (IL)

(72) Inventors: Emma KVITNITSKY, Kiryat Shemona (IL); Ram SNIR, Jerusalem (IL); Sanaa MUSA, Deir El-Assad (IL); Irena PALUY, Galil Elion (IL); Olga PRIVALOVA, Kiryat Yam (IL); Inna LITNETSKY, Karmiel (IL); Yehoyada BEERI, Moreshet (IL)

(73) Assignee: Nuversys LTD., Upper Galilee (IL)

(21) Appl. No.: 17/909,020

(22) PCT Filed: Mar. 1, 2021
(86) PCT No.: PCT/IL2021/050228

§ 371 Date: Sep. 2, 2022

Related U.S. Application Data
(60) Provisional application No. 62/983,919, filed on Mar. 2, 2020.

(51) Int. Cl. A61K 9/50; A23L 33/12

(52) U.S. Cl. CPC A61K 9/50; A23L 33/12

(57) ABSTRACT

Stable food-grade microcapsule designed to deliver a composition comprising at least one active substance to a food product; use of such microcapsules in the food industry; food products, food supplements, food articles and raw materials comprising such microcapsules are provided.

### FIELD OF THE INVENTION

The present invention relates in general to biologically active ingredients in food and/or food supplement products, and more accurately, to food and/or food supplement products comprising delivery system in form of microcapsules having improved properties.

### BACKGROUND OF THE INVENTION

Many biologically active substances used in food and/or food supplement products are unstable or incompatible with food due to their odor, taste or certain physical properties. Such substances therefore need to be incorporated into the food products in an “isolated” form, thus protecting them from external assaults and/or masking their undesired properties. In other words, the desired active substances should be delivered into the food without altering the performance of the food product while the activity/structure/nutritional value of the substance remains intact.

One of the most common examples of such substances are oxidizable oils containing polyunsaturated fatty acids. Over the past few decades, health experts have recommended diets rich in unsaturated fats. The unsaturated fatty acids play an essential role in the physiology of metabolic and structural processes in human body and have beneficial health effects. The biological actions of unsaturated fatty acids are manifold and produce a variety of therapeutic advantages. Extensive studies have implicated diverse abilities of unsaturated fatty acids to prevent coronary artery disease, associated with different mechanisms, including the deep involvement in eicosanoid biosynthesis to maintain physiological homeostasis and interaction with nuclear receptor proteins to modulate transcription of regulatory genes. Vegetable and marine oils, containing unsaturated fatty acids, are gaining increasing interest in the food industry because of their natural and safe status, wide acceptance by consumers, and multidimensional functional properties. The type and source of polyunsaturated acids such as Omega-3 and Omega-6 are as critical as their amount/concentration. The most common unsaturated oils from vegetable source are oleic acid, linoleic acid, α-linolenic and γ-linolenic acids, which lack the long chain fatty acids. Owing to large amount of omega-3 long-chain polyunsaturated fatty acids, marine and algal oils are in highest demand.

Fortification of food products with polyunsaturated fatty acids (PUFA) is seen as good alternative for increasing their intake, however, enrichment of foods with PUFA is technically challenging. This is especially true for foods prepared under high temperature processing conditions and/or are intended to have a relatively long ambient storage shelf life. Microencapsulation is one of the tools used to overcome the above challenges and is widely used in food industry. Spray drying is the most commonly used technology for microencapsulation of polyunsaturated fatty acids; however, some studies have also pointed out drawbacks of this technology. The simple spray-drying of an emulsion does not result in microcapsules suitable for use in food because of the low odor threshold of aroma-active compounds formed during the spray drying and further storage. The use of air as the drying medium at very high temperature produces particles with a porous structure. Even very small amounts of surface oil lead to offensive smell development. In addition, the spray dried powder particles can readily undergo oxidation, which decreases their shelf-life. For example, spray-dried fish oil powders are more susceptible to oxidation compared to the pure fish oil upon storage. In many cases, additional coating does not necessarily prevent malodor development and the type of coating material may significantly affect the sensory profile. Although spray-dried PUFAs powders have been successful in products such as bread and some others short shelf-life products, their stability in long shelf-life products remains poor. Extrusion technology leads to microcapsules having particle size in the range of 500-1000 μm, which is too large for inclusion in many food products. This due to the fact that particle size above 100 μm impacts the mouth feel. Complex coacervation technology also comes with some disadvantages. The coacervates formed with this technique are stable under very narrow range of pH. Current processes primarily use gelatin as the positively charged polymer, however animal derived gelatin is not acceptable to the vegetarian population and not acceptable for religious reasons.

Most encapsulation methods use water-soluble wall forming materials such as proteins, sugars, modified starches, gelatin and gums. However, these types of encapsulation are not suitable for protecting unsaturated fatty acids in food products that contain water or have a high-water activity because of dissolution and subsequent degradation of the encapsulated unsaturated fatty acids or oil sources upon contact with the food product. Since water is involved at one or more stages of processing and storage operations for most foods, encapsulation in water-soluble matrices has limited applicability for improving the stability of unsaturated fatty acids or for controlling retention and directed release of bioactive agents. It is therefore evident that improved methods for delivery of biologically active ingredients into the food products which are remain a long and unmet need.

### SUMMARY OF THE INVENTION

It is a principal object of the present invention to provide new and improved delivery systems of biologically active ingredient to the food products and food supplement product and their use in food industry.

The invention provides a stable food-grade microcapsule designed to deliver a composition comprising at least one active substance to a food product, wherein said at least one active substance is characterized by being incompatible with food and/or prone to degradation and/or having undesirable odor and/or taste; and wherein said microcapsule comprises a polymer shell and a core, wherein the shell is water and/or oil impermeable and made of an inert material, and wherein the composition comprising at least one active substance is enclosed inside the core of the microcapsule.

The invention further provides an article comprising a plurality of stable food-grade microcapsules designed to deliver a composition comprising at least one active substance to a food product, wherein said at least one active substance is characterized by being incompatible with food and/or prone to degradation and/or having undesirable odor and/or taste; and wherein said microcapsule comprises a polymer shell and a core, wherein the shell is water and/or oil impermeable and made of an inert material, and wherein the composition comprising at least one active substance is enclosed inside the core of the microcapsule.

The invention further provides a system for delivery of at least one active substance characterized by being incompatible with food and/or prone to degradation and/or having undesirable odor and/or taste to a food product for consumption, comprising at least one stable food-grade microcapsule designed to deliver a composition comprising at least one active substance to a food product, wherein said at least one active substance is characterized by being incompatible with food and/or prone to degradation and/or having undesirable odor and/or taste; and wherein said microcapsule comprises a polymer shell and a core, wherein the shell is water and/or oil impermeable and made of an inert material, and wherein the composition comprising at least one active substance is enclosed inside the core of the microcapsule.

The invention further provides a food product for consumption comprising an edible matter and an amount of stable food-grade microcapsules designed to deliver a composition comprising at least one active substance to a food product, wherein said at least one active substance is characterized by being incompatible with food and/or prone to degradation and/or having undesirable odor and/or taste; and wherein said microcapsule comprises a polymer shell and a core, wherein the shell is water and/or oil impermeable and made of an inert material, and wherein the composition comprising at least one active substance is enclosed inside the core of the microcapsule.

The invention further provides a process for the preparation of a food product enriched with at least one active substance characterized by being incompatible with food and/or prone to degradation and/or having undesirable taste and/or odor, said process comprising: a) providing a plurality of stable food-grade microcapsules of the invention; and b) introducing the plurality of stable food-grade microcapsules of the invention to the food product, to thereby obtain a food product enriched with the at least one active substance.

The invention further provides a food-grade raw material for the manufacture of a food product for consumption, wherein said raw material comprises an amount of stable food-grade microcapsules designed to deliver a composition comprising at least one active substance to a food product, wherein said at least one active substance is characterized by being incompatible with food and/or prone to degradation and/or having undesirable odor and/or taste; and wherein said microcapsule comprises a polymer shell and a core, wherein the shell is water and/or oil impermeable and made of an inert material, and wherein the composition comprising at least one active substance is enclosed inside the core of the microcapsule.

The invention further provides a plurality of food-grade stable microcapsules of the invention.

The invention further provides a device configured to store and/or release the plurality of food-grade stable microcapsules of the invention.

The invention further provides an assembly configured to release a predetermined amount of the plurality of food-grade stable microcapsules of the invention, said assembly comprising:

a. a housing; said housing comprising a container receiving chamber and a dispensing element, and b. a removable sealed container comprising the microcapsules, wherein said container is configured to be inserted into the container receiving chamber and to become operably engaged with the dispensing element;

wherein, when the container becomes operably engaged with the dispensing element, said dispensing element is configured to release the predetermined amount of the plurality of microcapsules from said sealed container.

The invention further provides a sealed container comprising the plurality of food-grade stable microcapsules of the invention, configured to be used with the assembly of the invention.

### BRIEF DESCRIPTION OF THE DRAWINGS

FIG. 1A illustrates an exemplary embodiment of an Optical microscope image of the 50% Omega-3 oil microcapsules with polymeric shell of ethyl cellulose at 10-× magnification;

FIG. 2 illustrates an exemplary embodiment of A. optical microscope image of 50% Omega-3 microcapsules with polymeric shell of Ethocel 100, at 10-× magnification; B. optical microscope image of 50% Omega-3 microcapsules with polymeric shell of Ethocel 45, at 10-× magnification; C. optical microscope image of 50% Omega-3 microcapsules with polymeric shell of Ethocel 10, at 10-× magnification;

FIG. 3 illustrates an exemplary embodiment of optical microscope image 50% Omega-3 microcapsules with polymeric shell of 10% zein and 40% ethyl cellulose, at 10-× magnification;

FIG. 4 illustrates an exemplary embodiment of A. SEM image of 50% Omega-3 microcapsules with polymeric shell comprises ethyl cellulose, at 760× magnification; B. SEM image of 50% Omega 3 microcapsules with polymeric shell comprises 20% shellac and 30% ethyl cellulose (Example 4), at 500-× magnification;

FIG. 5 illustrates an exemplary embodiment of A. release of Omega-3 oil from microcapsules of different types prepared accordingly to Example 1 and Example 2 in dissolution System 1, at pH 1.2 followed by dissolution in System 2, at pH 6.8; B. release of Omega-3 oil from microcapsules obtained with polymers of different chain length (Example 2) in dissolution System 1, at pH 1.2; C. Optical microscope image of intact microcapsules comprising 50% of Omega-3 oil at the start point (time zero) of the dissolution test, at 10× magnification; D. Optical microscope image of the microcapsules comprising 50% Omega-3 oil at the endpoint of the dissolution test in the System 2 (pH 6.8), at 10-× magnification;

FIG. 6 illustrates an exemplary embodiment of A. industrially produced gummies comprising microcapsules of Omega-3 oil. Each unit of gummy (3 g) contains 100 mg of DHA; B. optical microscope image of the dispersion of the Omega-3 oil microcapsules of the invention in gummies, at 10-× magnification; C. optical microscope image of the Omega-3 oil microcapsules of the invention isolated from gummies, at 10-× magnification;

FIG. 7 illustrates an exemplary embodiment of A. optical microscope image of the dispersion of microcapsules of the invention in yogurt, at 10-× magnification; B. optical microscope image of the dispersion of the microcapsules containing Omega-3 oil, which are produced by competitor, in yogurt, at 10-× magnification;

FIG. 8 illustrates an exemplary embodiment of optical microscope image of the dispersion of the Omega-3 oil microcapsules of the invention in the Healthy Bar, at 4-× magnification;

FIG. 9 illustrates an exemplary embodiment of A. SPME GC-MS chromatogram of the standards of secondary volatile metabolites of Omega-3 oil oxidation; B. content of secondary volatile metabolites of lipid oxidation in in microcapsules of the invention (Sample A) and in original Omega-3 oil (Sample B);

FIG. 10 illustrates an exemplary embodiment of A. optical microscope image of the microcapsules comprising 10% of Zinc Oxide, at 4-× magnification; B. SEM image of the microcapsules comprising 10% of Zinc Oxide, at 3700-× magnification; and

FIG. 11A. illustrates an exemplary embodiment of the assembly of the invention.

### DETAILED DESCRIPTION OF THE INVENTION

The present invention is now described more fully hereinafter with reference to the accompanying examples and drawings, in which embodiments of the invention are shown. This invention may, however, be embodied in many different forms and should not be construed as limited to the embodiments set forth herein; rather these embodiments are provided so that this disclosure will be thorough and complete and will fully convey the scope of the invention to those skilled in the art.

According to some embodiments, the invention provides a stable food-grade microcapsule designed to deliver a composition comprising at least one active substance to a food product, wherein said at least one active substance is characterized by being incompatible with food and/or prone to degradation and/or having undesirable odor and/or taste; and wherein said microcapsule comprises a polymer shell and a core, wherein the shell is water and/or oil impermeable and made of an inert material, and wherein the composition comprising at least one active substance is enclosed inside the core of the microcapsule. As used herein, the term “incompatible with food” is meant to be understood, without limitation as an active ingredient which is not suitable or impossible for use due to its odor, taste, color, or any other relevant parameter and/or characteristics. The term “active substance” as used herein, refers, without limitation, to any substance that provides health or any other benefits to the consumer. In one embodiment, the at least one active substance enclosed inside the core is isolated. In another embodiment, the at least one active substance enclosed inside the core retains its structure and/or biological activity. As used herein, the term “biological activity” refers, without limitation, to the capacity of a specific molecular entity to achieve a defined biological effect on a target and is measured in terms of potency or the concentration of the entity needed to produce the effect.

According to some embodiments, the microcapsule of the invention has a specific release profile. In one embodiment, upon consumption of the food product comprising the microcapsule, the at least one active substance is released from the microcapsule at the site of absorption and/or at the site of action. In another embodiment, the release profile of the at least one active substance is selected from a prolonged release profile, a delayed release profile, a sustained release profile, and an immediate release profile.

According to some embodiments, the at least one active substance comprises more than one biomolecule. In the context of the invention, the term “biomolecule” refers to any molecular entity having biological activity as defined above.

According to some embodiments, the microcapsule has the size of from 10 μm to 400 μm. In one embodiment, the microcapsule has the size of 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm; 90 μm; 95 μm, 100 μm, 110 μm, 115 μm, 120 μm, 125 μm, 130 μm, 135 μm, 140 μm, 145 μm, 150 μm, 155 μm, 160 μm, 165 μm, 170 μm, 175 μm, 180 μm, 185 μm, 190 μm, 195 μm, 200 μm, 210 μm, 215 μm, 220 μm, 225 μm, 230 μm, 235 μm, 240 μm, 245 μm, 250 μm, 255 μm, 260 μm, 265 μm, 270 μm, 275 μm, 280 μm, 285 μm, 290 μm, 295 μm, 300 μm, 310 μm, 315 μm, 320 μm, 325 μm, 330 μm, 335 μm, 340 μm, 345 μm, 350 μm, 355 μm, 360 μm, 365 μm, 370 μm, 375 μm, 380 μm, 385 μm, 390 μm, 395 μm, and 400 μm. The term “microcapsule”, as used herein, refers, without limitation, to a spherical microparticle consisting of a polymeric shell serving as a wall-forming material and encapsulated active ingredient/s within the core of the microcapsule.

According to some embodiments, the at least one active substance comprises a vitamin. A non-limiting list of the vitamins includes vitamin A, vitamin D, vitamin K, vitamin F and vitamin E, vitamins of group B, Coenzyme Q10, or a combination thereof.

According to some embodiments, the at least one active substance comprises natural and/or botanical extract or a derivative thereof. The non-limiting list of extracts includes Althea extract, Angelica extract, Anise extract, Arnica extract, Aronia extract, Astragalus extract, Basil extract, Cardamom extract, Chamomile extract, Celery seeds extract, Cloves extract, Cinnamon extract, Coriander extract, Cornsilk extract, Echinacea extract, Eucalyptus extract, Fennel extract, Garlic extract, Ginkgo Biloba extract, Ginseng extract, Ginger extract, Lemon grass extract, Licorice extract, Melissa extract, Mentha extract, Onion extract, Parsley extract, Passiflora extract, Pepper extract, Plantago extract, Rosemary extract, Thyme extract, Turmeric extract, Salvia extract, Sea-buckthorn extract, Hemp extract, Cannabis extract, Alaria extract, Bladderwrack extract, Dulse extract, Irish Moss extract, Kelp extract, Laminaria extract, layer extract, Rockweed extract, Sea Lettuce extract, Spirulina extract, and any combination thereof. In one embodiment, the at least one active substance is an isolate, individual compound.

According to some embodiments, the at least one active substance comprises a metal or a derivative thereof. A non-limiting list of metals and their derivatives includes ferrum or a derivative thereof, zinc or a derivative thereof, cupper or a derivative thereof, selenium a derivative thereof, and any combination thereof.

According to some embodiments, the at least one active substance is prone to oxidation. As used herein, the term “substance prone to oxidation” or “oxidizable substance”, are interchangeable and collectively refer to substance capable of undergoing a chemical reaction with oxygen, wherein substance is a form of matter that has constant chemical composition and characteristic properties. It cannot be separated into components without breaking chemical bonds. In one embodiment, the oxidizable substance comprises unsaturated and/or a polyunsaturated fatty acid. In one embodiment, the oxidizable substance is selected from the group consisting of fish oil, marine oil, krill oil, algal oil, vegetable oil, and plant oil. In another embodiment, the at least one active substance comprises at least one of: unsaturated omega-3 long-chain fatty acids, unsaturated omega-6 long-chain fatty acids, unsaturated omega-7 long-chain fatty acids, unsaturated omega-9 long-chain fatty acids, Eicosapentaenoic acid (EPA), Docosahexaenoic acid (DHA), Arachidonic acid, Trienoic fatty acids, alpha-Linolenic acid (ALA), polyunsaturated fatty acids (PUFAs) and any combination thereof. The term “Omega-3”, as used herein, refers, without limitation, to individual polyunsaturated fatty acids such as eicosapentaenoic acid (EPA), stearidonic acid (SDA), docosahexaenoic acid (DHA), alpha-linolenic acid (ALA), their esters and oils comprising such. The term “Omega-6”, as used herein, refers without limitation, to individual polyunsaturated fatty acids γ-linolenic acid (GLA), linoleic acid (LA), arachidonic acid (ARA), conjugated linoleic acid (CLA), and any oils comprising such. The terms “omega-3”, “omega-3 fatty acid(s)”, “omega-3 fat”, “omega-3 oil” and the like refer to omega-3 fatty acids as well as biologically relevant esters of these fatty acids including but not limited to triglycerides. These terms are also meant to encompass omega-3-containing oils (e.g., marine-derived oils and plant-derived oils), omega-3 fatty acids substantially purified from oils, and synthetically prepared omega-3. The term “Omega-7”, as used herein, refers, without limitation, to individual unsaturated fatty acids palmitoleic (9-hexadecenoic) acid, vaccenic (11-octadecenoic) acid, rumenic (octadeca-9,11-dienoic) acid, paullinic (13-eicosenoic) acid, and oils comprising such. The term “Omega-9”, as used herein, refers, without limitation to individual oleic acid and oils comprising such, and substantially purified from erucic acid. As used herein, the terms “omega-6”, “omega-6 fatty acid(s)”, “omega-6 fat”, “omega-6 oil” and the like refer to omega-6 fatty acids as well as omega-6 containing oils. The term “fish oil”, as used herein, refers, without limitation to the oil from any fish or fish part, or blends of oils from any fish or fish part, including but not limited to cod, cod liver, menhaden, sardines, salmon, anchovy, herring, and mackerel. The term “marine-derived”, as used herein, to the material was obtained from a marine animal such as fish, krill, plankton, or shellfish. The term “plant-derived”, as used herein, is meant to be understand as the material was obtained from a plant or plant part, such as seed, fruit, nut, or leaf.

According to some embodiments, the concentration of the at least one active substance enclosed inside the core is at least 5% by weight of the microcapsule. In one embodiment, the concentration of the at least one active substance inside the core is from 5% to 80% by weight of the microcapsule. In one embodiment, the concentration of at least one active substance enclosed inside the core is 5%, 10%, 15%, 20%, 25%, 30%, 35%, 40%, 45%, 50%, 55%, 60%, 65%, 70%, 75%, and 80% by weight of the microcapsule.

According to some embodiments, the undesirable taste and/or odor of the at least one active substance is essentially masked by the microcapsule. The term “essentially masked” is meant to be understood, without limitation, as a situation when undesirable odor and/or taste of the active substance is significantly reduced to fully eliminated.

According to some embodiments, the polymer of the shell is selected from a non-limiting list of polymers including Ethyl Cellulose, Cellulose Acetate Propionate, Cellulose Acetate, Carboxymethylcellulose, Carboxymethyl cellulose acetate butyrate, Hypromellose Acetate Succinate, Alginate and Alginate based polymers (for example, Aquateric™ N100), Zein, Casein, Whey proteins, Shellac, Carrageenan, Chitosan, Poly(L-lactide-co-glycolide), Cyclodextrins, Gum Arabic, Guar Gum, Xanthan Gum, Gum Ghatti, Gum Karaya, agar, furcellaran, Polylactide, poly-L-lactic acid (PLLA), poly-D-Lactic acid (PDLA), poly-D,L-lactic acid (PDLLA), Poly(ethylene glycol)-block-poly(D,L-lactic acid), Methoxy poly(ethylene glycol)-block-poly(D,L-lactic acid), or any combination thereof. In one embodiment, the polymer system is edible. In another embodiment, the polymer system is designed to release the microencapsulated active ingredient when ingested. In one embodiment, the polymer has a bland taste.

According to some embodiments, the core further comprises at least one antioxidant. The non-limiting list of antioxidants that can be used includes rosemary extract, rosmarinic acid, carnosic acid, anoxomer, carotenoids, BHT, BHA, and ascorbyl palmitate, or any other antioxidant that may be found suitable. According to some embodiments, the microcapsule further comprises at least one plasticizer. The non-limiting list of plasticizers that can be used includes coconut butter, cacao butter, paraffin oil, silicon oil, triglycerides of fatty acids, hydroxypropyl methylcellulose, acetyl triethyl citrate, triethyl citrate, triacetin, beeswax, candellila wax, carnauba wax, rice bran wax or any other plasticizer that may be found suitable.

According to some embodiments, the microcapsule further comprises at least one preservative. The non-limiting list of preservatives includes clove oil, oregano oil, rosemary oil, thyme oil, mustard oil, cinnamon oil, or individual antimicrobial compounds from this oil, for example 1,8-cineole, camphor, pinene, sodium benzoate, sodium nitrite, sulphur dioxide, sodium sorbate, potassium sorbate, or any other preservative that may be found suitable.

According to some embodiments, the microcapsule further comprises at least one flavoring agent. The non-limiting list of flavoring agents includes natural flavoring substances, or nature-identical flavoring substances such as citral, isoamyl acetate, benzaldehyde, cinnamic aldehyde, ethyl propionate, methyl anthranilate, limonene, ethyl decadienoate, allyl hexanoate, ethyl maltol, ethyl vanillin, methyl salicylate, or any other flavoring agent that may be found suitable.

According to some embodiments, the microcapsule further comprises at least one food colorant. A non-limiting list of food colorants of the invention includes Annatto, Carmine, Cochineal extract, Elderberry, Lycopene, Spirulina extract (Blue pigments), Paprika, Curcumin, Grape color extract, Canthaxanthin, Asthaxanthin, Anthocyanins, Dehydrated beets (beet powder), Beetroot extract, β-Apo-8′-carotenal, Carotenoids, Carrot oil, Brilliant Blue FCF, 5,5′-indigodisulfonic acid sodium salt (Indigo carmine), Fast Green FCF (N-ethyl-N-[4-[[4-[ethyl [(3-sulfophenyl)methyl]amino]phenyl](4-hydroxy-2-sulfophenyl)methylene]-2,5-cyclohexadien-1-ylidene]-3-ulfobenzenemethanaminium hydroxide), Erythrosine, Allura Red AC, Tartrazine, Sunset yellow FCF (disodium 2-hydroxy-1-(4-sulfonatophenylazo)naphthalene-6-sulfonate).

According to some embodiments, the invention provides article comprising a plurality of stable food-grade microcapsules of the invention. In one embodiment, the plurality of the stable food-grade microcapsules having an identical active substance content. In another embodiment, the article comprises a mixture of stable food-grade microcapsules having different active substance contents. In one embodiment, the article may be, without limitation, a dispersion, hard-shell capsule, soft gel capsule, syrup, juice, shot, solution, cream, shake, gummies, jelly, drink, mousse, butter, cake, bar, chewing gum, instant powder, powder, cocktail, pastille, chocolate, jam, peanut butter, paste, artificial meat, artificial fish, printed food product, and dairy product. As used herein, the term “printed food product” refers, without limitation to printed living cells that are incubated on a plant-based matrix to grow, differentiate and interact to achieve the texture and qualities of the real food. As used herein, the term “dairy product” refers, without limitation to type of food produced from or containing the milk of mammals, such as cattle, water buffaloes, goats, sheep, and camels. Dairy products include numerous food items such as yogurt, cheese and butter.

According to some embodiments of the invention, provided system for delivery of at least one active substance characterized by being incompatible with food and/or prone to degradation and/or having undesirable odor and/or taste to a food product for consumption, comprising at least one stable food-grade microcapsule of the invention. As used herein, the term “prone to degradation” refers, without limitation, to high vulnerability of matters to disintegration under various stress factors.

According to some embodiments, the invention provides a food product for consumption comprising an edible matter and an amount of stable food-grade microcapsules of the invention. In one embodiment, the food product is fortified food product. In one embodiment, the edible matter is in a liquid form. In another embodiment, the edible matter is in a solid form. In another embodiment, is in a semi-solid form. In one embodiment, the food product is a vegan product. In one embodiment, the food product is a vegetarian product. In one embodiment, the food product is a natural product. In one embodiment, the food product is from natural and/or plant source. As used herein, the term “natural product” refers, without limitation, to product produced by a living organism—that is, found in nature. In the broadest sense, natural products include any substance produced by life. Natural products may be obtained from cells, tissues, secretions of microorganisms, plants and animals. The term “natural source” as used herein, refers, without limitation to cells, tissues, secretions of microorganisms, plants and animals. In one embodiment, the food product is a functional food product. As used herein, the term “vegan product” refers, without limitation, to a product containing no animal ingredients or animal-derived ingredients. As used herein, the term “vegetarian product” refers, without limitation, to a product that meets vegetarian standards by not including meat and animal tissue products. As used herein, the term “semi-solid form” refers, without limitation to a state that is in between a solid and a liquid. Another name for a semi-solid is a quasi-solid. At the microscopic scale, it has a disordered structure unlike the more common solids.

According to some embodiments, provided a process for the preparation of a food product enriched with at least one active substance characterized by being incompatible with food and/or prone to degradation and/or having undesirable taste and/or odor, said process comprising: a) providing a plurality of stable food-grade microcapsules of the invention; and b) introducing the plurality of stable food-grade microcapsules of any one of the invention to the food product; to thereby obtain a food product enriched with the at least one desirable active ingredient.

According to some embodiments, provided a raw material for the manufacture of a food product for consumption, wherein said raw material comprises an amount of stable food-grade microcapsules of the invention. In one embodiment, the raw material is vegan. In one embodiment, the raw material is vegetarian. In one embodiment, the raw material is a food-grade premix for the manufacture of a food product for consumption, wherein said premix comprises a desirable amount of stable food-grade microcapsules of the invention. As used herein, the term “premix” refers, without limitation, to a blend of food-grade components that has been mixed in advance of use or of further processing.

According to some embodiments, the invention provides a plurality of food-grade stable microcapsules of the invention.

The microcapsules of the invention can be used in many ways, including, without limitation, ready-to-use product and/or packaging, a ready-to-eat food, packaging and product for manual application by the end user, a device designed to be used by the end-user for application of the microcapsules to the desired food in order achieve even particle distribution and/or specific, predetermined dosing. The device can be manual, automatic, or semi-automatic.

According to some embodiments the device can be, without limitation, a volumetric bottle and/or box and/or package and/or container; a sachet; a spraying bottle and/or box, a dispenser, or any other device and/or packaging means suitable for storage and application of the microcapsules of the invention into the desired food as a single dose and/or multidose.

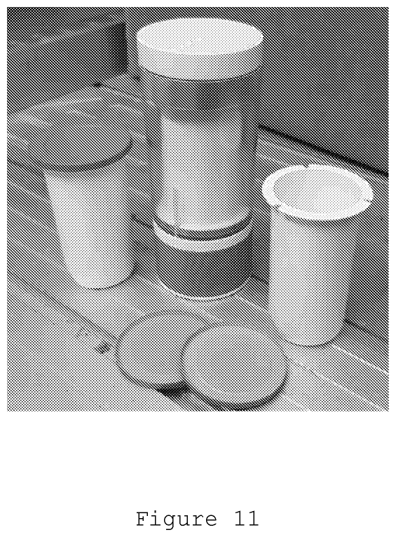

According to some embodiments, the invention provides an assembly configured to release a predetermined amount of the plurality of food-grade stable microcapsules of the invention, said assembly comprising a. a housing; said housing comprising a container receiving chamber and a dispensing element, and b. a removable sealed container comprising the microcapsules, wherein said container is configured to be inserted into the container receiving chamber and to become operably engaged with the dispensing element; wherein, when the container becomes operably engaged with the dispensing element, said dispensing element is configured to release the predetermined amount of the plurality of microcapsules from said sealed container. Reference is now made to FIG. 11 illustrating an exemplary embodiment of the assembly of the invention. The assembly of the invention is a dispenser designed to release a desired amount of microcapsules of the invention.

The container receiving chamber is designed to accommodate a container, in such way, that once the container inserted into the container receiving chamber it becomes attached to the dispersing element. The dispersing element, may be operated in any appropriate way and any suitable mechanical mechanism which allows to effectively release the desirable amount of the microcapsules. For example, the release of the microcapsules may be operated by a rotation of the dispensing element. The dispenser of the invention may contain a tool designed to open the sealed container, once the container is inserted into the container receiving chamber. The dispenser or its parts can be made of any suitable material known in the art. The container-receiving chamber of the dispenser can accommodate a container of a particular size and/or shape. Alternatively, the container-receiving chamber of the dispenser may be adjustable, and thus being used with a plurality of containers.

According to some embodiments, the invention provides a sealed container comprising the plurality of food-grade stable microcapsules of the invention and configured to be used with the assembly of the invention. The container of the invention can be made of any suitable material. The container or its parts can be made of biodegradable material. The container nay be reusable.

Disclosed herein are food articles, which comprise microencapsulated omega-3 of the present invention. The microcapsules containing a loading substance is to be delivered to a consumer upon eating or drinking the food article. The disclosed food articles can be any article that can be consumed (e.g., eaten, drank, or ingested) by a consumer. It can be desired that the food article be a palatable and popular food article. By using food articles that are widely accepted, compliance with dietary or dosage regimens for the loading omega-3 can be increased. Exemplary food articles include but are not limited to nutrition bars, chocolate, baked goods such as cookies, crackers, pies, snack cakes, bread, and doughs. The food product could be provided as ready-to-drink beverage or in dry form to be reconstituting with liquid for drinking. The food product could be yogurt, cereal, cheese, or other type of hand-held food products. Preferably, the food are a nutrition bar and dairy products. In various preferred embodiments, the present invention relates to the release of bioactive agents. During the food processing, storage the end food product and then upon ingestion of the food or beverage products by human, the delivery system remains intact and stable. The delivery system of the present invention allows reaching release of active substances at the site of absorption of these active substances. The delivery system does not substantially dissociate in the acidic environment of gastric fluid of the stomach (pH is typically in the range of 1.5-3.5). The delivery system substantially releases the bioactive agents in the small intestine (lower gastrointestinal tract, pH>6) in a pH-controlled manner, thus enhancing bioavailability and overall physiological efficacy of the microencapsulated bioactive agent. Desired amount of unsaturated fatty acids microcapsules is provided to the food products described herein. The amount to be added varies to suit a particular application and can be based, at least in part, on taste, shelf-life, nutritional value, efficacy levels approved, qualified health claims, and combinations thereof. For example, it may be desired to provide at least 32 mg of omega-3 fatty acids (combined EPA and DHA) per serving of the food product, or at least 300 mg omega-3 fatty acids per serving of the food product to meet the United States Food and Drug Administration (FDA) nutrient content claim requirements. The unsaturated fatty acids microcapsules are sufficiently mixed in the food product to provide a relatively uniform distribution; however, mixing is not limited to suspending of the unsaturated fatty acids microcapsules in a food formulation. For example, the unsaturated fatty acids may be mixed in powder form with a powdered drink mix or powdered milk (e.g., Incolac®, Nesquik®, or caffeinated drink mix GFuel®) to form a substantially evenly blended powdered product. In the practice of the present invention any of omega-3, omega-6, omega-7 and omega-9 fatty acids and oil-sources of unsaturated fatty acids such as flaxseed oil, olive oil, walnut oil, macadamia oil, sea buckthorn oil, borage oil, sunflower oil, soybean oil, cashew nut oil, peanut oil, avocado oil, marine oils or blend thereof may be used. Marine oils include but not limiting to anchovies, herring, sardines, menhaden, salmon, trout, and mackerel and krill oils. Microcapsules of the invention may comprise the mixture of omega-3 and omega-6 fatty acids in ratio 1:4, most preferably in ratio 1:1. The present invention relates to a method for producing a food product. The method includes the steps of pre-processing to form an intermediate food product (a premix), adding a desired amount of microencapsulated unsaturated fatty acids to the intermediate food product, and mixing the intermediate food product to disperse the unsaturated fatty acids in the intermediate food product. Optionally, the method can include the steps of pasteurizing the intermediate food product to form a food product and post-processing the food product. Post-processing may include preparing the product for packaging. The intermediate food product could be a solution or a semi-solid or solid mixture. The present invention relates to a food product including product mixture and a desired amount of microencapsulated unsaturated fatty acids dispersed in the product mixture by mixing the product mixture could be a solid mixture or semisolid or a liquid. The adding step may include adding microencapsulated unsaturated fatty acids to the intermediate food product by, for example, using a powder mixing. The mixing step may include dispersing the microencapsulated unsaturated fatty acids within the intermediate food product to form a substantially homogenous blend using for example shear mixer. Other methods provided herein involve mixing the microcapsules with one or more ingredients used in the process of preparing a food item, prior to its production. Alternative or additional methods include contacting the finished food product with the microcapsules. For example, the microcapsules may be mixed with seasonings for a food item. The above methods are not limited to any particular method of adding microcapsules to the pre-homogenized composition. For example, the microcapsules can be manually introduced or poured into the pre-homogenized composition. Alternatively, the microcapsules may be pumped into pre-homogenized compositions or added via a feed hopper. Other suitable methods for adding delivery vehicles to the pre-homogenized composition are known in the art. The mixing can also be carried out by methods known in the art, such as, but not limited to, mechanical agitators, magnetic agitators, shakers, devices for mixing with gas, mixing with ultrasound, shaking, etc. When using microencapsulated unsaturated fatty acids, these compositions can be incorporated into food products without significant destruction in the process of obtaining food products. In particular, the microcapsules of the present invention resistant to breakage during the production of a food item (including packaging, transportation and storage of a food item). The microcapsules have a size and texture that does not make the texture and consistency of the food product not attractive. The finished food product of present invention including unsaturated fatty acids microcapsules may have a long shelf-life of about 2-12 months and possibly up to 24 months under ambient conditions, depending on the level of processing the product undergoes, the type of packaging, and the materials used for packaging the product. Microencapsulation method can be based on solvent removal process. An exemplary method for production of microcapsules for application in food products comprises the steps of: a) dissolving or dispersing the unsaturated fatty acids, optionally together with an antioxidant, a plasticizer, a flavor, a preservative, other additives or mixture thereof in an Ethyl Acetate that is partially miscible with water and is capable of dissolving or dispersing said substances, together with a wall-forming polymer selected from the group consisting of Ethyl Cellulose, Cellulose Acetate Propionate, Cellulose Acetate, Carboxymethylcellulose, Carboxymethyl cellulose acetate butyrate, Hypromellose Acetate Succinate, Alginate and Alginate based polymers (for example, Aquateric™ N100), Zein, Casein, Whey proteins, Shellac, Carrageenan, Chitosan, Poly(L-lactide-co-glycolide), Cyclodextrins, Gum Arabic, Guar Gum, Xanthan Gum, Gum Ghatti, Gum Karaya, agar, furcellaran, Polylactide, poly-L-lactic acid (PLLA), poly-D-Lactic acid (PDLA), poly-D,L-lactic acid (PDLLA), Poly(ethylene glycol)-block-poly(D,L-lactic acid), Methoxy poly(ethylene glycol)-block-poly(D,L-lactic acid), or a combination thereof to form an organic solution; b) preparing an aqueous continuous phase saturated with said organic solvent and comprising an emulsifier; c) while agitating, pouring the organic solution or dispersion obtained in (a) into the aqueous continuous phase obtained in (b), to form an emulsion; d) adding an excess amount of water to the emulsion obtained in (c) to initiate extraction of the organic solvent from the emulsion, and optionally incubating for further removing of the solvent and formation of solid microcapsules (hereinafter “the core microcapsules”); the excess amount of water is generally an excess of about 20:1; e) immersing the core microcapsules into an aqueous solution of alcohol, separating the core microcapsules and drying at temperature not exceed 200C, thus obtaining single-layer micro capsules. In one embodiment, the polymer of the inner core microcapsule and of the outer shells may be identical or different. The second layer covering single-layer microcapsules can be achieved also by using combination of solvent removing method with coacervation, fluidized bed or inclusion into cyclodextrins. This additional barrier coating enables modifying properties of the delivery system and provides programmed release. The microcapsules of the present invention are intended for application in food products. Such a use requires a unique design of the microcapsules, with respect to their mechanical properties. Microcapsules have to be hard enough to avoid destruction of shell and realization of the content during technological process knowing in the art of food production. Such mechanical properties are achieved by choosing an appropriate wall forming material. In addition, selection of a suitable plasticizer and determining its percentage are another important factor. The plasticizer may be selected from natural oils and fats (e.g. cacao butter, coconut butter, avocado oil), silicon oils, paraffin oil, triacetin, triethyl citrate, acethyl triethyl citrate, triglycerides of fatty acids (e.g. trilaurin, tricapsylin, tripalmitin), hydroxypropyl methyl cellulose, various waxes (e.g. beeswax, candellila, carnauba and rice bran waxes) and mixture thereof. The presence of the plasticizer in the microcapsules of the present invention affects their mechanical properties thus positively affecting their use and efficiency. Such emulsifying agents may be used either alone or in combination thereof. The concentration of the plasticizing agent may be selected from the range of about 1% to about 10% and is preferably in the range of about 1% to about 6%. The microcapsules of the invention may further comprise at least one antioxidant. Examples of antioxidants suitable for the present disclosure include, but are not limited to, a-tocopherol (vitamin E), calcium disodium EDTA, alpha tocopheryl acetates, butylhydroxytoluenes (BHT), and butylhydroxyanisoles (BHA), CoQ10, anoxomer. Other examples of antioxidants include ascorbic acid and pharmaceutically acceptable salts thereof such as sodium ascorbate, pharmaceutically acceptable esters of ascorbic acid including fatty acid ester conjugates, propyl gallate, citric acid and pharmaceutically acceptable salts thereof, malic acid and pharmaceutically acceptable salts thereof. Further non-limiting examples of antioxidants include natural antioxidants, such as, for example, plant-derived extracts or oils, such as Rosmarinus (Rosemary), Oreganum, Thymus, and Artemisice (Tarragon), and/or individual natural compounds, such as lutein, zeaxanthan, β-carotene. The antioxidants can be used in an amount of from 1 to 10% by weight of the final microcapsules. Microcapsules of the invention may further comprise at least one preservative. The preservatives may be selected from plant-derived essential oils such as clove oil, oregano oil, rosemary oil, thyme oil, mustard oil, cinnamon oil, or individual antimicrobial compounds from this oil, for example 1,8-cineole, camphor, pinene and etc. Preservatives are also may be selected from sodium benzoate, sodium nitrite, sulphur dioxide, sodium sorbate, potassium sorbate. Microcapsules of the invention may further comprise at least one flavor agent selected from natural flavoring substances, or nature-identical flavoring substances such as citral, isoamyl acetate, benzaldehyde, cinnamic aldehyde, ethyl propionate, methyl anthranilate, limonene, ethyl decadienoate, allyl hexanoate, ethyl maltol, ethyl vanillin, methyl salicylate. In the second stage, an aqueous continuous phase is saturated by the Ethyl Acetate and an appropriate emulsifier may be added to the aqueous phase. Examples of the emulsifying agents that may be used include, without limitation, poly(vinyl alcohol), polyvinyl pyrrolidone, carboxymethyl cellulose, sodium carboxymethyl cellulose, lauryl phosphate, ethoxylated sorbates such as Tween-20 and Tween-60, polyglycerol and poly(ethylene glycol), and their esters and ethers, and the like. Such emulsifying agents may be used either alone or in combination thereof. The concentration of the emulsifying agent may be selected from the range of about 0.1% to about 10% and is preferably in the range of about 0.1% to about 5%. In order to remove trace amounts of the solvent, the microcapsules obtained after filtration are immersed in a 10% solution of ethanol in thereby causing the Ethyl Acetate residue to be removed from the microcapsules. Under such conditions the Ethyl Acetate residue present within the microcapsules migrates from the microcapsules to the outer medium rapidly, and the remaining trace of solvent, less than 5000 ppm in the microcapsules, is well within the allowed FDA range. The process of the present invention is easy scalable. On an industrial scale production, after separation of microcapsules, the organic phase can be removed from the water phase by distillation. Thus, both the water and organic phase can be recycled. The microcapsules described here, as a rule, combine in themselves a high payload, i.e. high percentage of omega-3 oil per gram of the microcapsules powder, and structural strength. For example, the described microcapsules are strong enough to withstand the process of homogenization. In addition, the content of unsaturated fatty acids in the described microcapsules can be from about 20 to about 80%, from about 50 to about 80%, or about 60% by weight of the microcapsule. Prevention of oxidation during the formation of microcapsules can be achieved by carrying out the process under vacuum, in the presence of an inert gas, protected from light and/or under sterile conditions. The formed microcapsules for human consumption, should resist the usual alimentary industry processes, in particular to operations knowing in the art. The microcapsules of the invention can be subjected to unit operations such as: sterilization, homogenization, pasteurization, ozonation, use of chemical antimicrobial products (either natural or synthetic). The microbiological stabilizers can be added in the industrial process, therefore, in a particular embodiment, in the interior of the microcapsules and/or the phase of food formulation that contain the microcapsules, it is found a stabilizer material in terms of microbiological quality. The microcapsules of the present invention may have particularly good shelf-life stability and taste when reconstituted in milk or other liquid dairy food products. The microcapsules of the present invention are characterized in terms of morphology, size and size distribution, oil encapsulation efficiency and pH sensitivity.

### Example 1: Preparation of Microencapsulated Omega-3 Algal Oil in Ethyl Cellulose

20 g of the microcapsules comprising 50% Omega-3 algal oil were prepared. To an aqueous continuous phase of 260 ml of tap water saturated with 40 ml of ethyl acetate polyvinyl alcohol (PVA) was added as an emulsifier. An organic phase was prepared from 10 g ethyl cellulose in 100 ml of ethyl acetate. Then, 10 g of Omega-3 algal oil, and 0.2 g of BHT and 0.2 g of cacao butter were added to the organic phase and the mixture was stirred at room temperature to get homogenous solution. The resulting organic phase was poured into the aqueous phase, while stirring at 400-450 rpm for 30 min to form homogenous emulsion. This emulsion was poured into 4 liter of water; the obtained mixture was stirred at 400 rpm for 20 minutes and then kept overnight at room temperature. The microcapsules were filtrated and washed, and were then air-dried at the temperature not higher than 20° C. An average diameter of the microcapsules was 80-150 μm. Efficiency of encapsulation was 94%.

Results:

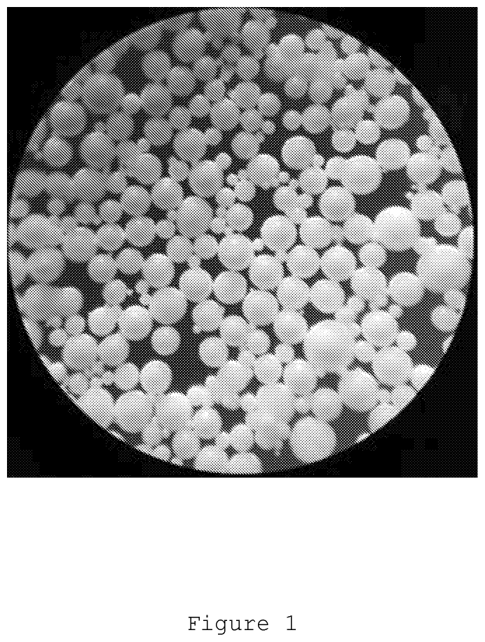

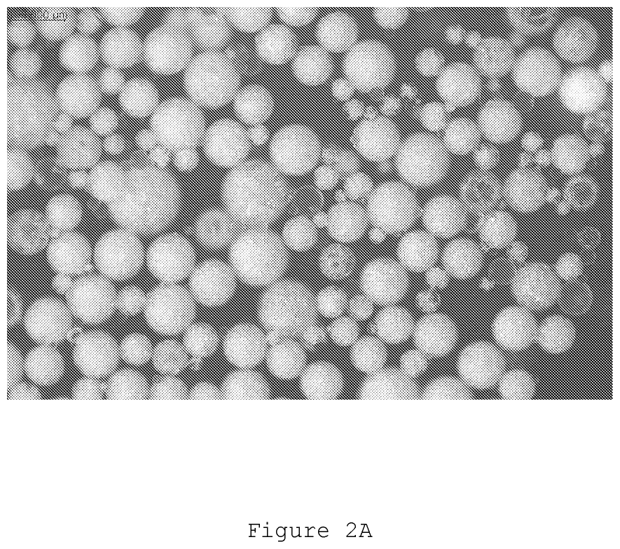

The obtained microcapsules contained 50% of Omega-3 algal oil. FIG. 1 demonstrates microscope image of the 50% Omega-3 oil microcapsules with polymeric shell of ethyl cellulose.

### DISCUSSION

By the same manner, any oil-soluble active ingredient can be microencapsulated. In the result, these microcapsules will increase stability and shelf life of the oil and prevent development of distinct malodor during the storage of the product.

### Example 2: Impact of Polymers with Different Chain Length on Properties of Microcapsules

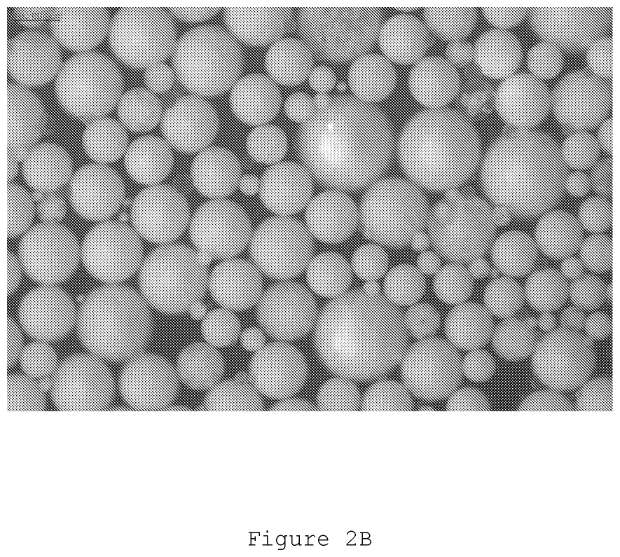

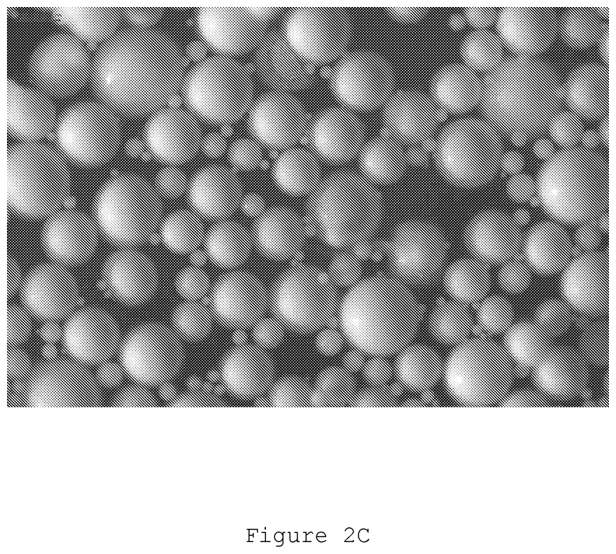

A. Polymers of ethyl cellulose with different chain length were evaluated for encapsulation of 50% Omega-3 algal oil. Ethocel 10, Ethocel 20, Ethocel 45 and Ethocel 100 (DuPont) were used. Preparation of the capsules was carried in the same manner as in the Example 1. The obtained microcapsules differ morphologically and consequently by properties. Increase in polymer chain length reduces the smoothness of the capsule surface. FIG. 2A-2C shows optical microscope images of three type of microcapsules.

B. Four types of these microcapsules were incorporated into gummy formulation for evaluation of the organoleptic properties. 11% of microcapsules of Omega-3 oil were incorporated into pectin heated up to 75° C. and stirred for 3 min, and then 0.02 g of 50% water solution of citric acid was added and stirred. The content of DHA per one unit of gummy 3 g was calculated as 100 mg. The formulation was transferred to starch powder and was kept for drying at room temperature overnight.

Results:

The tested parameters were: a) sensation of microcapsules while eating gummies, b) masking Omega-3 oil original odor and taste in gummies. A proprietary sensory screening method, the ASTM E1627-19, Standard Practice for Sensory Evaluation of Edible Oils and Fats, was used. The panel of seven trained tasters carried out the sensory evaluation. The level of hardness of microcapsules was determined for polymers as Ethocel 100>Ethocel 45>Ethocel 20>Ethocel 10. In case of Ethocel 10 no sensation of microcapsules in gummy was determined. In addition, typical Omega-3 oil taste neither specific Omega-3 oil odor were determined for all polymers used. Images of microcapsules are presented by FIG. 2A-D.

### Example 3: Preparation of Microencapsulated Omega-3 Algal Oil in the Composition of Ethyl Cellulose and Zein

10 g of the microcapsules comprising 50% of Omega-3 algal oil were prepared with composition of polymers comprising 40% of ethyl cellulose and 10% of zein.

An aqueous continuous phase was 120 ml of tap water saturated with 20 ml of ethyl acetate and added PVA as emulsifier. An organic phase was prepared from 4 g ethyl cellulose, 5 g of Omega-3 oil in 40 ml of ethyl acetate and 10 g of 10% zein solution in 85% aqueous ethanol, the mixture was stirred to get homogenous dispersion. The resulting organic phase was poured into the aqueous phase, while stirring for 30 min to form homogenous emulsion. This emulsion was transferred into 2.0 liter of water. The obtained mixture was stirred at 200 rpm for 20 minutes at room temperature. The microcapsules were filtrated. Then microcapsules were transferred into 60 ml of cold 10% aqueous ethanol, stirred and filtrated again. The microcapsules were dried in vacuum oven. The yield of this process is 87%. Particle size—80-200 micron.

Results

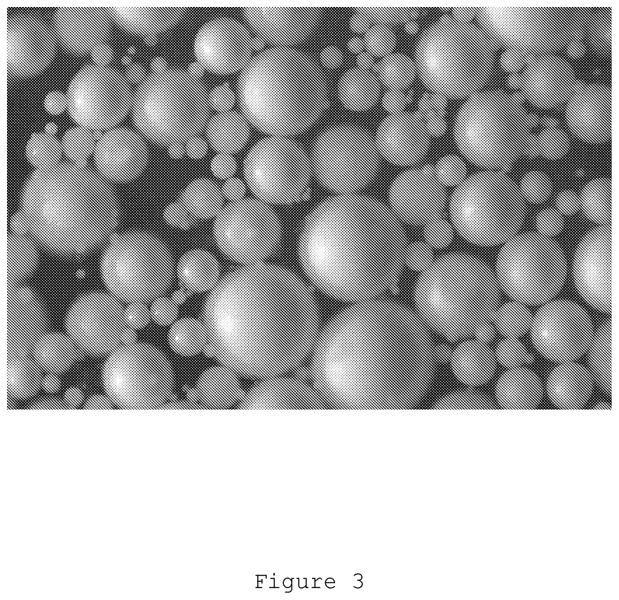

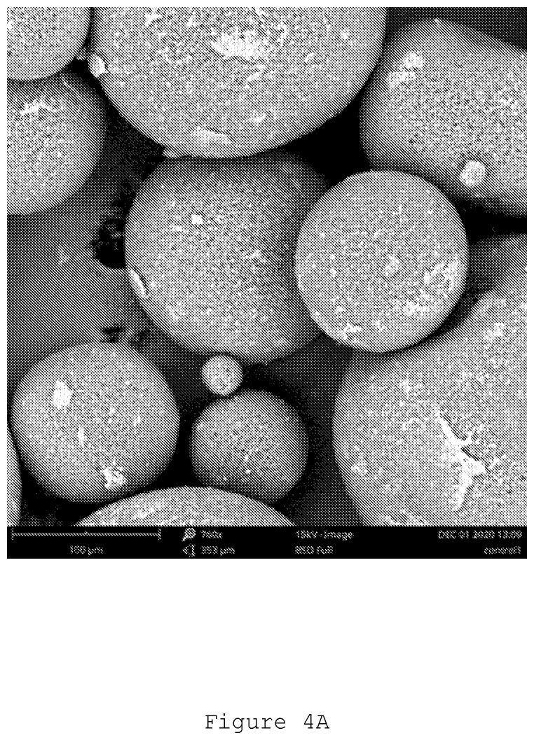

Addition of zein allows obtaining the microcapsules with smoother surface then capsules obtained in Examples 1 and 2. FIG. 3 demonstrate optical microscope image of the microcapsules with zein.

### Example 4: Preparation of Microencapsulated Omega-3 Algal Oil in the Composition of Polymers of Ethyl Cellulose and Shellac

20 g of composite microcapsules comprising 50% of Omega-3 algal oil were prepared with 30% of ethyl cellulose and 20% Shellac. An aqueous continuous phase was 200 ml of tap water saturated with 30 ml of ethyl acetate and added PVA as emulsifier. An organic phase was prepared from 6 g Ethyl Cellulose, 10 g Omega-3 algal oil in 70 ml of ethyl acetate and 20 ml of ethanol solution of 4 g dewaxed Shellac. The mixture was and stirred to full dissolution. The resulting organic phase was poured into the aqueous phase while stirring for 30 min to obtain uniform emulsion and then emulsion was transferred into 2.51 of water. The microcapsules were filtrated under vacuum, placed in cold 10% aqueous ethanol, stirred for 10 min, filtrated again and dried in vacuum oven. The yield of this process is 95%. Particle size—100-200 micron.

Results

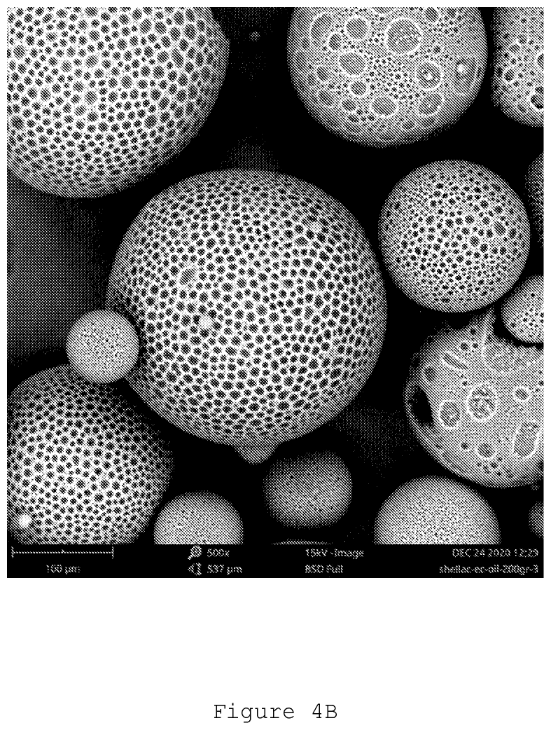

Morphology and properties (hardness, fragility, profile of release, ability of masking taste/odor and protection of Omega-3 oil) of these microcapsules were found as principally different from microcapsules obtained in Examples 1, 2 and 3. The use of shellac as enteric coating material allows creating capsules with programmed realizing properties to be tailored for specific products. FIG. 4A demonstrates SEM image of microcapsules comprising 50% of Omega-3 algal oil enveloped in ethyl cellulose (Example 1). FIG. 4B demonstrates SEM image of microcapsules comprising 50% Omega-3 enveloped in 30% of ethyl cellulose and 20% of shellac.

### Example 5: Release of Omega-3 Oil from Microcapsules

To evaluate the release of Omega-3 oil from microcapsules, USP <711>dissolution modified test was applied. To improving the efficiency of the release of oil, 0.25% of Tween 20 was added to the dissolution medium. Two dissolution media for the release test were used: System 1-0.1M Hydrochloric acid, pH 1.2; System 2-0.1M Phosphate buffer, pH 6.8.

Experiment A

Two samples were tested: the microcapsules of Example 1 (Prototype 1) and the microcapsules of Example 4 (Prototype 2. 200 mg of microcapsules were stirred at 200 rpm in 900 ml of dissolution medium of System 1 at 37.5° C. After 3 hours, the microcapsules were filtrated, then washed with distillated water. Microcapsules were air-dried and then content of Omega-3 remained in capsules was measured by UV spectrophotometry. The samples were prepared by dissolving about 10-15 mg of microcapsules in 1 ml Methanol in 25-ml volumetric flask and then filling to volume with n-Hexane and measured at 210 nm. Based on the amount of remained Omega-3 oil in capsules the extent of release of oil in Sytem 1 was calculated. The release in System 1 was about 18% after 3 hour of experiment for both types of tested capsules.

The capsules filtrated from System 1 were transferred into 900 ml of dissolution medium of System 2 and were stirred for additional 3 hours at 37.5° C. The microcapsules were filtrated, washed with distillated water and air-dried. Content of Omega-3 oil remained in microcapsules was measured as described herein above.

Results

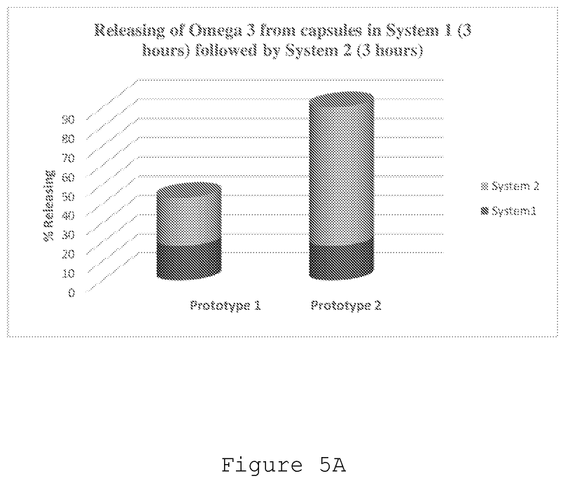

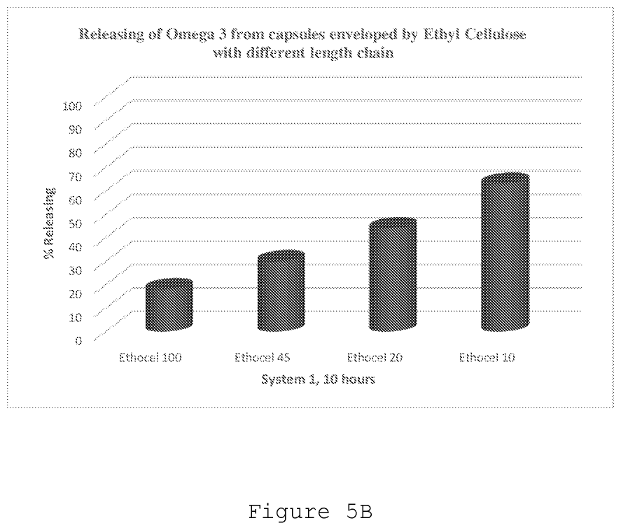

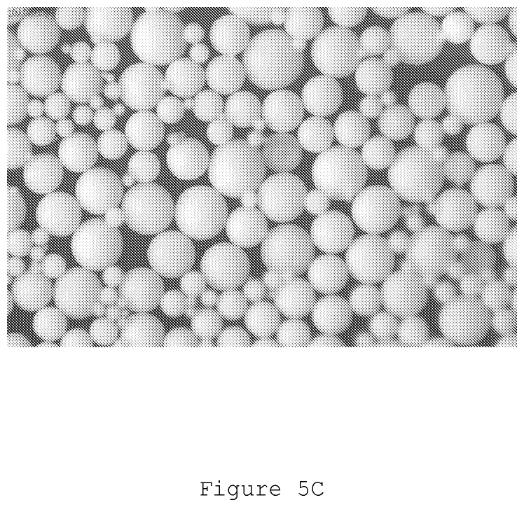

The release of Omega-3 oil encapsulated in ethyl cellulose (Prototype 1) in System 2 was 25%, whereas the release of Omega-3 oil from composite microcapsules (Prototype 2) was 72%. The comparison between release of Omega-3 oil from microcapsules of Prototype1 and Prototype 2 are given in FIG. 5A.

### DISCUSSION

Use of entering polymers (like as shellac) in microcapsules' polymeric wall allow controlling release of Omega-3 oil from microcapsules of the invention at the intended site of human body

Experiment B:

Release of Omega-3 oil from microcapsules obtained accordingly to Example 2 with Ethyl Cellulose of different chain length. The microcapsules were prepared with Ethocel 100, Ethocel 45, Ethocel 20 and Ethocel 10. The extent of release was evaluated during 10 hours using System 1 as described in the Example 5A.

Results

The representative graphs of released Omega-3 from four types of microcapsules obtained using polymers of different chain length is given in FIG. 5B. Increase of the chain length of ethyl cellulose demonstrated different level of release and depending of the level of release on chain length of polymer.

### DISCUSSION

The process of the release of the Omega-3 oil can be demonstrated by microscopy of the microcapsules before, after the test as described in the Example 5A. The microcapsules were prepared in the manner described in Example 4 (Prototype 2). The intact capsules before releasing are shown in FIG. 5C. After releasing in both System 1 and System 2, the capsules appears empty as shown in FIG. 5D.

### Example 6: Gummies Comprising Microencapsulated Omega-3 Oil

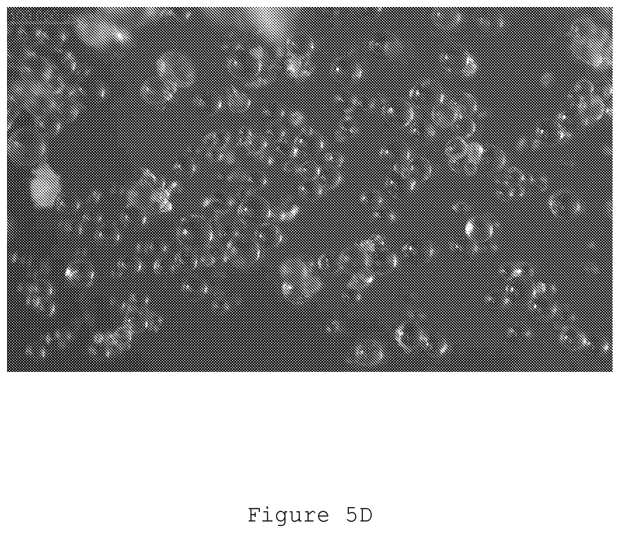

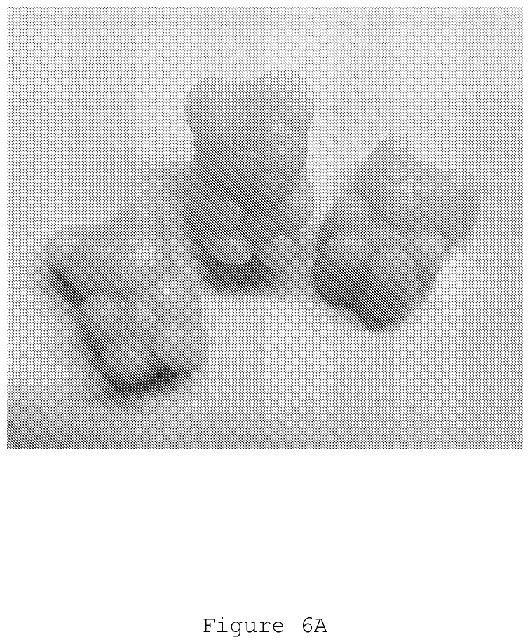

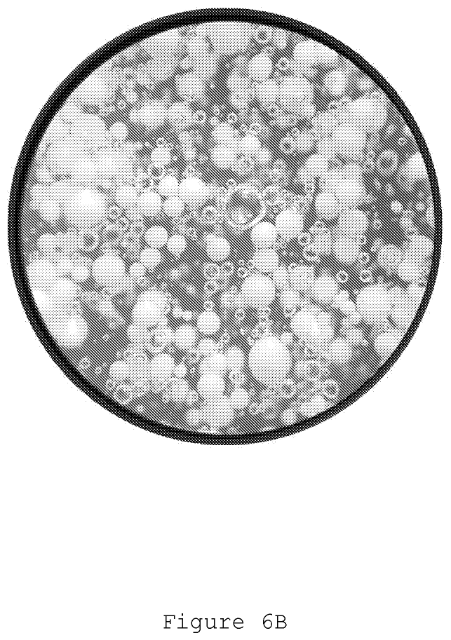

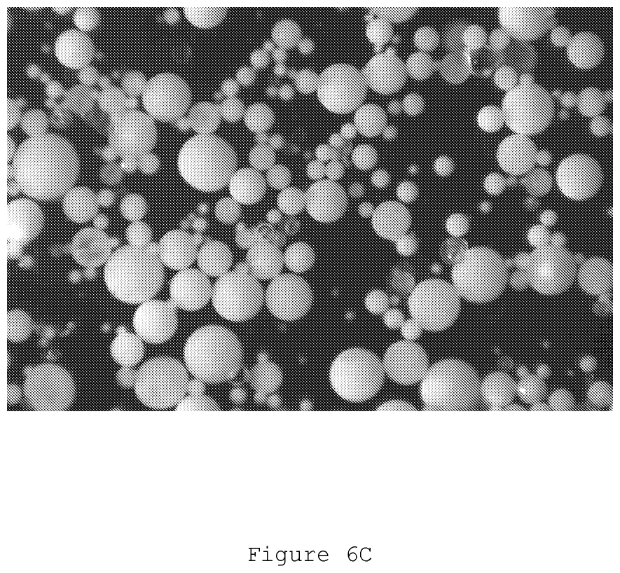

Gummies comprising the microcapsules of Omega-3 oil (Example 1) were produced on the industrial scale. The content of DHA per one unit of gummy 3 g was calculated as 100 mg. Gummy image is presented in the FIG. 6A. Under microscopic examination, it was found that the microcapsules that incorporated into the gummies, retained their original shape, and saved and protected Omega-3 oil inside the capsules as shown in the FIG. 6B. For studying the integrity and content, the microcapsules were isolated from gummies into water. Isolated and dried microcapsules were tested for content of Omega-3 oil using UV spectroscopy according to procedure described above in the Example 5. The amount of Omega-3 oil remained in microcapsules isolated from gummies was 97-98%. The intact capsules isolated from gummies kept the original shape, kept and protect Omega 3 inside the capsules as demonstrated in FIG. 6C.

### Example 7: Yogurt Comprising Microencapsulated Omega-3 Oil

The 11% of microcapsules of Omega-3 oil obtained in Example 3 were incorporated into a commercially available yogurt. The content of DHA per 1 package of commercial yogurt (200 g) was calculated as 100 mg. After two weeks of storage in refrigerator (4° C.) the sensory and organoleptic properties of yogurt were tested. No offensive sensation of microcapsules, typical odor and taste of Omega-3 oil were detected in sample of yogurt comprising the microcapsules of the invention.

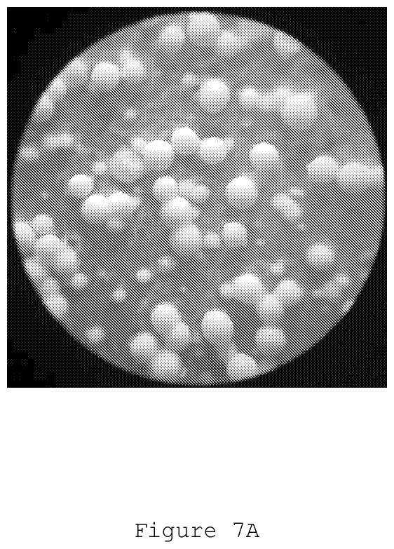

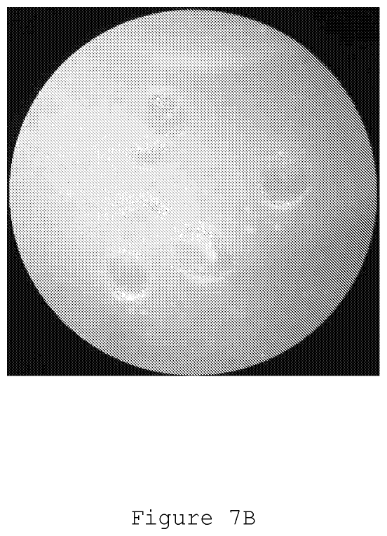

Microcapsules comprising Omega-3 oil produced by different technology and purchased from market (competitor's sample) was in the other portion of the same yogurt. The pronounced differences were demonstrated using microscopy observation while comparing the samples (FIGS. 7A and 7B).

Results

The microcapsules of the invention kept original shape as well as Omega-3 inside the capsules without leaching into the yogurt, whereas the microcapsules produced by other technology of microencapsulation (by competitor) partially dissolved and released Omega-3 oil into the yogurt, as proven by the organoleptic test.

### Example 8: Healthy Bar Comprising Microencapsulated Omega-3 Oil

Healthy Bars comprising the microcapsules of Omega-3 oil (Example 4) were produced on the industrial scale. The content of DHA per one unit of healthy bar (30 g) was calculated as 250 mg. The formulation for healthy bars was prepared from chopped dates, raisins, chopped walnut or cashew nuts, sesame seeds, sunflower seeds. These ingredients were mixed with microencapsulated Omega-3 at 50° C. for two minutes. The distribution of Omega-3 oil microcapsules healthy bar is shown in FIG. 8.

Results

Under microscopic examination, it was found that the microcapsules retained their original shape and protected Omega-3 oil inside of the capsules.

### Example 9: Analysis of Secondary Volatile Lipid Oxidation Products Via HS-SPME/GC-MS

Stability of microencapsulated Omega-3 oil was tested using solid phase microextraction (SPME) combined with gas chromatography-mass spectrometry (GC-MS). This method served to identify and quantify the volatile organic compounds (VOC) formed due to the degradative oxidation. The majority of VOC from the decomposition reaction of unsaturated fatty acids, such as aldehydes, possess low odor thresholds. Oxidation level of an oil can be defined based on the presence of different chemical species, especially Popanal, 2-Pentenal, 3-Hexanal, 2,4-Heptadienal, 1-Penten-3-one and 1-Penten-3-ol. Appearance of these species may define the rancidity and malodor development of microencapsulated Omega-3. Mixture of standards (Merck) in concentration of 25, 50 and 100 ppm were prepared in n-Hexane. The microcapsules of the invention, Sample A, were compared with original Omega-3 algal oil, which was used as raw material for preparation of these capsules, Sample B. Sample A was prepared accordingly the procedure given in Example 1. Both samples were stored at room temperature for 1 month. The SPME-fiber (DVB/CAR/PDMS; Divinylbenzene/Carboxen/polydimethylsiloxane, Supelco) was placed in the headspace of 20 ml vials, which were filled with 500 mg of microcapsules samples comprising 250 mg of Omega-3 oil or with 250 μL of original Omega-3 oil, or with 200 μl of standards mixture and sealed with a butyl rubber/PTFE-septum. The extraction was performed at 45° C. for 40 minutes. The analytes were desorbed from the SPME fiber at 250° C. for 180 s. The injection was splitless and the whole system remained at a constant flow of 2 mL/min with helium as carrier gas. Separation was performed on DB-624 cyanopropyl phenyl/polydimethylsiloxane capillary column (30 m×0.32 mm×0.2 μm). The temperature program was as follows: after holding at constant temperature of 40° C. for 5 minutes, the temperature was raised by 2° C./min up to a temperature of 60° C. After 2 minutes isothermal hold, the temperature was increased up to 120° C. at a rate of 10° C./min. Finally, the temperature was increased up to 260° C. at a heating rate of 40 C/min. The final temperature was held for 10 minutes. Transfer line temperature was set to 280° C. and electron ionization mass spectrometry was performed at 70 eV. After one minute of solvent delay all ions between m/z 35-300 were plotted (SCAN mode) and typical fragments of the analyzed standards were recorded in selected ion mode. Each standard was injected individually and the relative retention times were calculated. In addition, the spectra were compared to those in the NIST98 and Wiley spectra library. Then mixture of standard was injected. The retention time (Rt) of each analyte is given in Table 1:

| Compound name | Retention time | SIM Mass (m/z) |
|---|---|---|
| Propanal | 4.25 | 57, 58 |
| 1-Penten-3-one | 8.32 | 55, 84 |
| Hexanal | 9.94 | 44, 56, 72, 82 |
| E-2-Pentenal | 11.35 | 55, 83, 84 |
| 1-Penten-3-ol | 12.45 | 57, 86 |
| 2,4-Heptadienal | 22.62 | 81, 110 |

Results

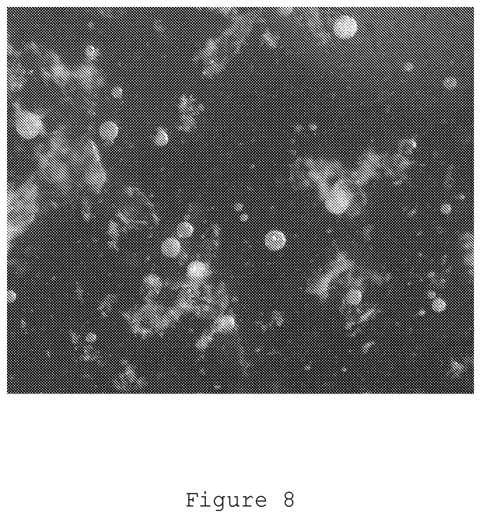

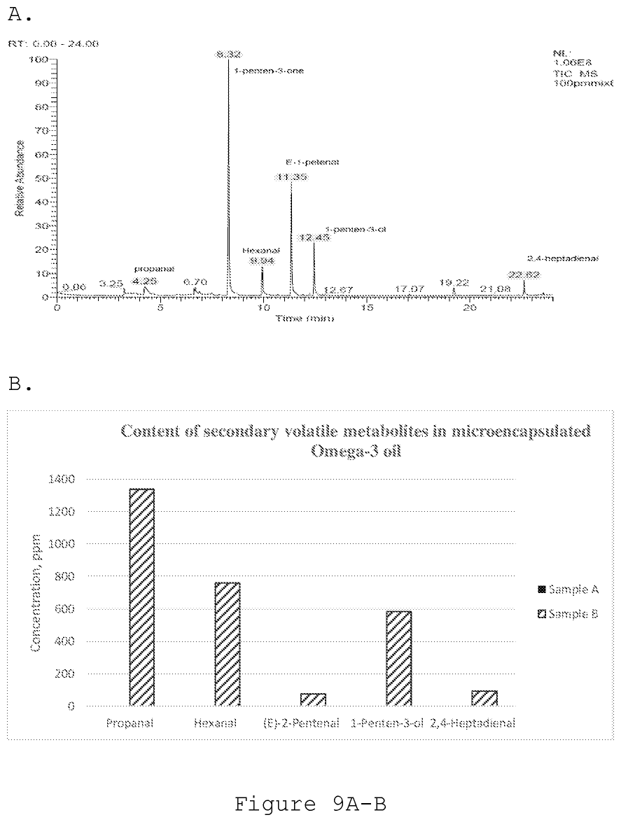

The chromatogram of standards is present in the FIG. 9A. The graph in the FIG. 9B presents the content of the secondary volatile metabolites of lipid oxidation in sample A and sample B. As shown in the table below, only four analytes were determined in both samples—Hexanal, E-2-Pentenal, 1-Penten-3-ol and 2,4-Heptadienal. Propanal was determined only in the aged Sample B. Analyte 1-Penten-3-one was not found in any of the samples, as shown in Table 2:

| | Peak Area | | Content, ppm | |
|---|---|---|---|---|
| Analyte | Sample A | Sample B | Sample A | Sample B |
| Propanal | Not detected | 745602515.4 | 0 | 1338.6 |
| 1-Penten-3-one | Not detected | Not detected | 0 | 0 |
| Hexanal | 61375.791 | 417855244.6 | 0.11 | 759.7 |
| (E)-2-Pentenal | 105791.461 | 134915953.2 | 0.06 | 75.9 |
| 1-Penten-3-ol | 144053.609 | 500980545.7 | 0.17 | 583.3 |
| 2,4-Heptadienal | 215195.65 | 19534777.8 | 1.03 | 93.1 |

The results are well correlated with the organoleptic odor analysis, which has demonstrated the absence of malodor of the Sample A. The invented microcapsules effectively protected Omega-3 oil from oxidative degradation and subsequently from malodor development.

### Example 10: Preparation of Microencapsulated Zinc Oxide in Ethyl Cellulose

The microencapsulation of Zinc oxide as potential antiviral agent may allow incorporating into chewing gum and further sustained release in mouth cavity.

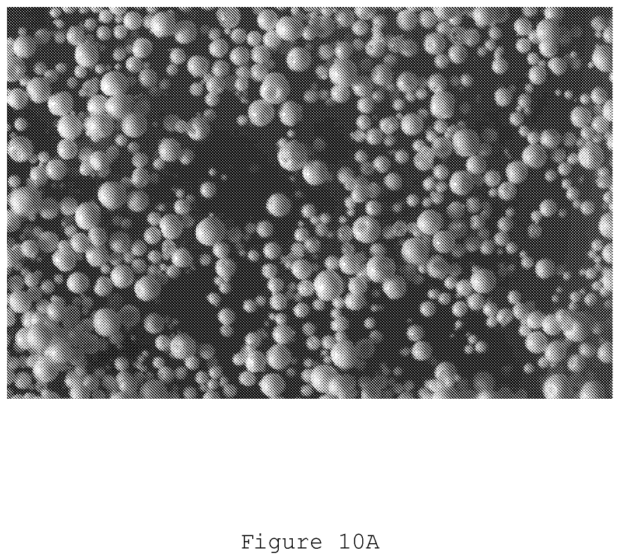

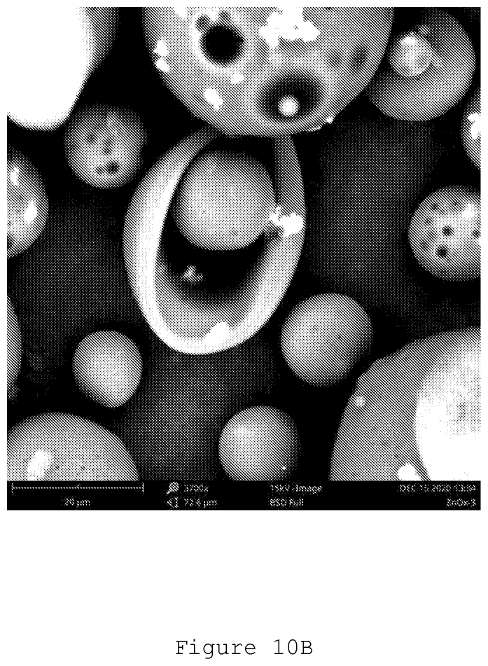

60 g of microcapsules comprising 10% of Zinc oxide were prepared. An aqueous continuous phase was prepared from 1400 ml of tap water saturated with 200 ml of ethyl acetate and PVA as emulsifier. An organic phase was prepared from 45 g ethyl cellulose and 9 g of Triacetin as plasticizer in 550 ml of ethyl acetate by stirring until complete dissolution. Then suspension, prepared separately, of 6 g of zinc oxide in 50 ml of ethyl acetate was added to organic phase. The resulting organic phase was poured into the aqueous phase while stirring for 30 min to form homogenous emulsion. This emulsion was poured into 15 Liter of water; obtaining mixture was stirred and then kept overnight at room temperature. After decantation of liquid, containing water and ethyl acetate, remaining suspension of microcapsules was filtrated under vacuum and dried in the vacuum oven. The yield of this process is 95%. Particle size—50-80 micron. FIG. 10A shows microscope image of the microcapsules comprising Zinc Oxide. FIG. 10B shows SEM image of the microcapsules comprising Zinc Oxide.

### Example 11: Preparation of Microencapsulated Cinnamon Oil in Ethyl Cellulose

Cinnamon food-grade oil possesses antibacterial and antiviral activity. The microencapsulation of Cinnamon oil allows odor and test masking effects and allowing incorporating microcapsules comprising Cinnamon oil into chewing gum or other products without negative impact of processing on quality and stability of oil.

20 g of the microcapsules comprising 10% of Cinnamon oil were prepared. An aqueous continuous phase was prepared from 260 ml of tap water saturated with 40 ml of ethyl acetate and PVA added as emulsifier. An organic phase was prepared from 18 g ethyl cellulose and 2 g of Cinnamon oil in 100 ml of ethyl acetate by stirring at until complete dissolution. The resulting organic phase was poured into the aqueous phase, while stirring to form homogenous emulsion. This emulsion was poured into 3 liter water. The obtained mixture was stirred at for 30 minutes and then kept overnight. The microcapsules were filtrated and dried in vacuum oven. The yield of this process is 96%. Particle size—80-200 micron.

### Example 12: Preparation of Microencapsulated Hemp Oil in Ethyl Cellulose

Hemp seed oil is growing in popularity because it provides a long list of health benefits that have been confirmed through an ongoing body of research. In addition to CBD (cannabidiol), hemp oil contains large amounts of omega-6 and omega-3 fats and all nine essential amino acids. Like all oils, hemp oil is vulnerable to heat, air, and light that can cause oxidation and alter the efficacy of the oil. Microencapsulation protects hemp oil from unwanted environmental and processing influences.

20 g of the microcapsules comprising 50% of Hemp oil were prepared. An aqueous continuous phase consisted from 260 ml of tap water saturated with 40 ml of ethyl acetate and PVA added as emulsifier. An organic phase was prepared from 10 g ethyl cellulose, 10 g of hemp oil and 0.2 g of tricalcium phosphate in 100 ml of ethyl acetate by stirring until complete dissolution. The resulting organic phase was poured into the aqueous phase, while stirring at room temperature to form homogenous emulsion. This emulsion was transferred into 4 liter of water; obtaining mixture was stirred for 30 minutes and then kept overnight. After careful decantation of mixture of water and ethyl acetate, remained suspension of microcapsules were filtrated and dried in vacuum oven. The yield of this process is 94%. Particle size — 80-150 micron.

The terminology used herein is for the purpose of describing particular embodiments only and is not intended to be limiting of the invention. As used herein, the singular forms “a,” “an” and “the” are intended to include plural forms as well, unless the context clearly indicates otherwise. It will be further understood that the terms “comprises” or “comprising,” when used in this specification, specify the presence of stated features, integers, steps, operations, elements components and/or groups or combinations thereof, but do not preclude the presence or addition of one or more other features, integers, steps, operations, elements, components and/or groups or combinations thereof. As used herein the terms “comprises”, “comprising”, “includes”, “including”, “having” and their conjugates mean “including but not limited to”. The term “consisting of” means “including and limited to”.

As used herein, the term “and/or” includes any and all possible combinations or one or more of the associated listed items, as well as the lack of combinations when interpreted in the alternative (“or”).

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and claims and should not be interpreted in an idealized or overly formal sense unless expressly so defined herein. Well-known functions or constructions may not be described in detail for brevity and/or clarity.

It will be understood that, although the terms first, second, etc., may be used herein to describe various elements, components, regions, layers and/or sections, these elements, components, regions, layers and/or sections should not be limited by these terms. Rather, these terms are only used to distinguish one element, component, region, layer and/or section, from another element, component, region, layer and/or section.

It will be understood that when an element is referred to as being “on,” “attached” to, “operatively coupled” to, “operatively linked” to, “operatively engaged” with, “connected” to, “coupled” with, “contacting,” etc., another element, it can be directly on, attached to, connected to, operatively coupled to, operatively engaged with, coupled with and/or contacting the other element or intervening elements can also be present. In contrast, when an element is referred to as being “directly contacting” another element, there are no intervening elements present.

Certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Throughout this application, various embodiments of this invention may be presented in a range format. It should be understood that the description in range format is merely for convenience and brevity and should not be construed as an inflexible limitation on the scope of the invention.

Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Whenever the term “about” is used, it is meant to refer to a measurable value such as an amount, a temporal duration, and the like, and is meant to encompass variations of ±20%, ±10%, +5%, ±1%, or ±0.1% from the specified value, as such variations are appropriate to perform the disclosed methods.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

All publications, patent applications, patents, and other references mentioned in the disclosures of these publications in their entireties are hereby incorporated by reference into this application in order to more fully describe the state of the art to which this invention pertains. In case of conflict, the patent specification, including definitions, will prevail.

In addition, the materials, methods, and examples are illustrative only and not intended to be limiting. Throughout this application various publications, published patent applications and published patents are referenced.

It will be appreciated by persons skilled in the art that the present invention is not limited to what has been particularly shown and described hereinabove. Rather the scope of the present invention is defined by the appended claims and includes both combinations and sub-combinations of the various features described hereinabove as well as variations and modifications thereof, which would occur to persons skilled in the art upon reading the foregoing description. While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention. Various embodiments have been presented. Each of these embodiments may of course include features from other embodiments presented, and embodiments not specifically described may include various features described herein.

1. A stable food-grade microcapsule designed to deliver a composition comprising at least one active substance to a food product, wherein said at least one active substance is characterized by being incompatible with food and/or prone to degradation and/or having undesirable odor and/or taste; and wherein said microcapsule comprises a polymer shell and a core, wherein the shell is water and/or oil impermeable and made of an inert material, and wherein the composition comprising at least one active substance is enclosed inside the core of the microcapsule.

2. (canceled)

3. The microcapsule of claim 1, wherein the at least one active substance enclosed inside the core retains its structure and/or biological activity.

4. The microcapsule of claim 1, having a specific release profile, wherein said profile is selected from a prolonged release profile, a delayed release profile, a sustained release profile, and an immediate release profile.

5. The microcapsule of claim 1, wherein upon consumption of the food product comprising the microcapsule, the at least one active substance is released from the microcapsule at the site of absorption and/or at the site of action.

6. (canceled)

7. The microcapsule of claim 1, wherein the at least one active substance is prone to oxidation; and wherein the at least one active substance comprises at least one of unsaturated fatty acids, polyunsaturated fatty acids, vitamins, metals or derivatives thereof, or a combination thereof.

8. (canceled)

9. The microcapsule of claim 7, wherein the at least one active substance comprises at least one of unsaturated omega-3 long-chain fatty acid, unsaturated omega-6 long-chain fatty acid, unsaturated omega-7 long-chain fatty acids, unsaturated omega-9 long-chain fatty acids, Eicosapentaenoic acid (EPA), Docosahexaenoic acid (DHA), Arachidonic acid, Trienoic fatty acids, alpha-Linolenic acid (ALA), polyunsaturated fatty acids (PUFAs), vitamin A, vitamin D, vitamin E, vitamin K, vitamin F, vitamins of group B, Coenzyme Q10, ferrum or a derivative thereof, zinc or a derivative thereof, cupper or a derivative thereof, selenium or a derivative thereof, or any combination thereof.

10. The microcapsule of claim 11, wherein the at least one active substance is selected from the group consisting of fish oil, marine oil, krill oil, algal oil, vegetable oil, and plant oil.

11. (canceled)

12. (canceled)

13. (canceled)

14. (canceled)

15. The microcapsule of claim 1-7, wherein the at least one active substance comprises a natural and/or botanical extract or a derivative thereof.

16. The microcapsule of claim 15, wherein said extract is selected from the group consisting of Althea extract, Angelica extract, Anise extract, Arnica extract, Aronia extract, Astragalus extract, Basil extract, Cardamom extract, Chamomile extract, Celery seeds extract, Cloves extract, Cinnamon extract, Coriander extract, Cornsilk extract, Echinacea extract, Eucalyptus extract, Fennel extract, Garlic extract, Ginkgo Biloba extract, Ginseng extract, Ginger extract, Lemon grass extract, Licorice extract, Melissa extract, Mentha extract, Onion extract, Parsley extract, Passiflora extract, Pepper extract, Plantago extract, Rosemary extract, Thyme extract, Turmeric extract, Salvia extract, Sea-buckthorn extract, Hemp extract, Cannabis extract, Alaria extract, Bladderwrack extract, Dulse extract, Irish Moss extract, Kelp extract, Laminaria extract, layer extract, Rockweed extract, Sea Lettuce extract, Spirulina extract, and any combination thereof.

17. The microcapsule of claim 1, wherein the concentration of the at least one active substance enclosed inside the core is at least 5% by weight of the microcapsule.

18. (canceled)

19. The microcapsule of claim 1, wherein the undesirable taste and/or odor of the at least one active substance is essentially masked by the microcapsule.

20. (canceled)

21. The microcapsule of claim 1, wherein the polymer of the shell is selected from the group consisting of Ethyl Cellulose, Cellulose Acetate Propionate, Cellulose Acetate, Carboxymethylcellulose, Carboxymethyl cellulose acetate butyrate, Hypromellose Acetate Succinate, Alginate and Alginate based polymers, Zein, Casein, Whey proteins, Shellac, Carrageenan, Chitosan, Poly(L-lactide-co-glycolide), Cyclodextrins, Gum Arabic, Guar Gum, Xanthan Gum, Gum Ghatti, Gum Karaya, agar, furcellaran, Polylactide, poly-L-lactic acid (PLLA), poly-D-Lactic acid (PDLA), poly-D,L-lactic acid (PDLLA), Poly(ethylene glycol)-block-poly(D,L-lactic acid), Methoxy poly(ethylene glycol)-block-poly(D, L-lactic acid), or a combination thereof.

22. The microcapsule of claim 1, further comprising at least one of: at least one antioxidant, at least one flavoring agent, at least one colorant or a combination thereof.

23. (canceled)

24. (canceled)

25. (canceled)

26. (canceled)

27. (canceled)

28. (canceled)

29. The microcapsule of claim 1, having the size of from 10 μm to 400 μm.

30. An article comprising a plurality of stable food-grade microcapsules of claim 1, wherein said plurality of stable food-grade microcapsules having an identical or a different content.

31. (canceled)

32. (canceled)

33. The article of claim 30 selected from the group consisting of dispersion, hard-shell capsule, soft gel capsule, syrup, juice, shot, solution, cream, shake, gummies, jelly, drink, bar, chewing gum, instant powder, powder, cocktail, pastille, chocolate, jam, peanut butter, paste, artificial meat, artificial fish, printed food product, and dairy product.

34. The article of claim 30, wherein said article is a food supplement.

35. A system for delivery of at least one active substance characterized by being incompatible with food and/or prone to degradation and/or having undesirable odor and/or taste to a food product for consumption, comprising at least one stable food-grade microcapsule of claim 1.

36. A food product for consumption comprising an edible matter and an amount of stable food-grade microcapsules claim 1.

37. The food product of claim 36, wherein the edible matter is in a liquid form, a solid form, or a semi-solid form, and, optionally, wherein said product is selected from a fortified food product, a functional food product, a vegan product, a vegetarian product, a natural product, or any combination thereof.

38. (canceled)

39. (canceled)

40. (canceled)

41. (canceled)

42. (canceled)

43. (canceled)

44. A process for the preparation of a food product enriched with at least one active substance characterized by being incompatible with food and/or prone to degradation and/or having undesirable taste and/or odor, said process comprising: a) providing a plurality of stable food-grade microcapsules of claim 1; and b) introducing the plurality said microcapsules to the food product, to thereby obtain a food product enriched with the at least one active substance.

45. A food-grade raw material for the manufacture of a food product for consumption, wherein said raw material comprises an amount of stable food-grade microcapsules of claim 1, and, optionally, wherein said raw material is selected from a vegan raw material or a vegetarian raw material.

46. (canceled)

47. (canceled)

48. (canceled)

49. (canceled)

50. A device configured to store and/or release a plurality of food-grade stable microcapsules of claim 1 wherein said device is selected from the group consisting of a volumetric bottle, a volumetric container, a volumetric package, a sachet, a spraying container, a spraying bottle, and a dispenser.

51. (canceled)

52. (canceled)

53. An assembly configured to release a predetermined amount of a plurality of food-grade stable microcapsules of claim 1, said assembly comprising:
a. a housing; said housing comprising a container receiving chamber and a dispensing element, and
b. a removable sealed container comprising the microcapsules, wherein said container is configured to be inserted into the container receiving chamber and to become operably engaged with the dispensing element.
wherein, when the container becomes operably engaged with the dispensing element, said dispensing element is configured to release the predetermined amount of the plurality of microcapsules from said sealed container.

54. A sealed container comprising the plurality of food-grade stable microcapsules of claim 49, configured to be used with the assembly of claim 53.
